(19) United States
(12) Patent Application Publication
(10) Pub. No.: US 2023/0320943 A1
(43) Pub. Date: Oct. 12, 2023

(54) Oral Care Compositions and Methods for the Same

(71) Applicant: Colgate-Palmolive Company, New York, NY (US)

(72) Inventors: Guisheng PAN, Philadelphia, PA (US); Gregory SZEWCZYK, Flemington, NJ (US); Suman CHOPRA, Monroe, NJ (US)

(73) Assignee: Colgate-Palmolive Company, New York, NY (US)

(21) Appl. No.: 18/296,782

(22) Filed: Apr. 6, 2023

Related U.S. Application Data
(60) Provisional application No. 63/328,978, filed on Apr. 8, 2022.

(51) Int. Cl. A61K 8/02; A61K 8/22; A61K 8/23; A61K 8/81; A61K 8/24; A61K 8/73; A61K 8/34; A61K 8/46; A61K 8/44; A61Q 11/00

(52) U.S. Cl. CPC A61K 8/0216; A61K 8/22; A61K 8/23; A61K 8/8176; A61K 8/24; A61K 8/731; A61K 8/345; A61K 8/463; A61K 8/44; A61Q 11/00; A61K 2800/30; A61K 2800/805; A61K 2800/594

(57) ABSTRACT

Solid oral care compositions and methods for the same are disclosed. The oral care composition may include an orally acceptable vehicle and one or more whitening agents. The oral care composition may be a toothpaste in the form of a solid film. The one or more whitening agents may include at least one of a source of hydrogen peroxide, a non-peroxide bleaching agent, or a combination thereof. The method for preparing the oral care composition may include contacting the orally acceptable vehicle, the one or more whitening agents, and water with one another to prepare a slurry, and preparing the solid film with the slurry.

### CROSS-REFERENCE TO RELATED APPLICATIONS

This application claims the benefit of U.S. Provisional Patent Application No. 63/328,978, filed on Apr. 8, 2022, which his incorporated herein by reference to the extent consistent with the present disclosure.

### BACKGROUND

Conventional oral care products including whitening agents are often utilized to whiten teeth. For example, toothpastes often include peroxides, such as hydrogen peroxide, to oxidize chromophores bound to surfaces and/or dentin of teeth to thereby whiten the teeth. While the incorporation of whitening agents in conventional oral care products, such as toothpaste, has proven to be effective for whitening teeth, the incorporation of these whitening agents in novel oral care products has proven to be difficult due to the general instability and/or degradation of the whitening agents. For example, oral cleansing and whitening compositions in the form of solid films have been developed. However, the incorporation of stable whitening agents in these solid films has presented challenges due to the instability of the whitening agents and/or the degradation of the whitening agents.

What is needed, then, are improved solid films of the oral cleansing and whitening compositions including one or more whitening agents and method for the same.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing an oral care composition including an orally acceptable vehicle and one or more whitening agents. The oral care composition may be a toothpaste in the form of a solid film. The one or more whitening agents may include at least one of a source of hydrogen peroxide, a non-peroxide bleaching agent, or a combination thereof. The solid film may be dissolvable and/or disintegrable.

In at least one implementation, the whitening agent may include the source of hydrogen peroxide.

In at least one implementation, the source of hydrogen peroxide may include a hydrogen peroxide solution.

In at least one implementation, the source of hydrogen peroxide may be a solid. The source of hydrogen peroxide may include one or more of a cross-linked polyvinylpyrrolidone (PVP) hydrogen peroxide complex, a polyvinylpyrrolidone (PVP) hydrogen peroxide complex, or a combination thereof.

In at least one implementation, the source of hydrogen peroxide may be present in an amount sufficient to provide free hydrogen peroxide in an amount of from about 0.01 weight % to less than or equal to about 10 weight %, based on the total weight of the oral care composition.

In at least one implementation, the whitening agent may include the non-peroxide bleaching agent.

In at least one implementation, the non-peroxide bleaching agent may include a salt of monopersulfate (MPS).

In at least one implementation, the salt of MPS may include one or more of potassium MPS, sodium MPS, ammonium MPS, or a combination thereof.

In at least one implementation, the salt of MPS may include 2KHSO₅·KHSO₄·K₂SO₄.

In at least one implementation, the non-peroxide bleaching agent may be present in an amount of from greater than 0 weight % to less than or equal to about 15 weight %.

In at least one implementation, the orally acceptable vehicle may include a polymer base including one or more water soluble polymers.

In at least one implementation, the one or more water soluble polymers may include one or more of carboxymethyl cellulose (CMC), hydroxyethyl cellulose (HEC), cetyl hydroxyethylcellulose, hydroxypropyl methylcellulose (HPMC), hydroxypropyl cellulose (HPC), polyvinylpyrrolidone (PVP), polyvinylpyrrolidone/vinyl acetate (PVP/VA), cellulose ethers, or a combination thereof.

In at least one implementation, the one or more water soluble polymers may include polyvinylpyrrolidone (PVP).

In at least one implementation, the PVP may include a combination of high molecular weight PVP and low molecular weight PVP.

In at least one implementation, the high molecular weight PVP may include a molecular weight of from about 200,000 Da to about 1,500,000 Da, or about 300,000 Da to about 1,300,000 Da, or from 500,000 Da to about 1,000,000 Da.

In at least one implementation, the low molecular weight PVP may include a molecular weight of from about 10,000 Da to about 100,000 Da, about 30,000 Da to about 70,000 Da, or about 40,000 Da to about 60,000, Da.

In at least one implementation, a weight ratio of the high molecular weight PVP to the low molecular weight PVP may be from about 0.5:1 to about 2:1.

In at least one implementation, the oral care composition further includes one or more plasticizers.

In at least one implementation, the plasticizer includes one or more of glycerin, sorbitol, an alkylene glycol, or a combination thereof.

In at least one implementation, the plasticizer includes the alkylene glycol. The alkylene glycol may include one or more of polyethylene glycol, polypropylene glycol, or a combination thereof.

In at least one implementation, the alkylene glycol may include polyethylene glycol in an amount of from about 4 weight % to about 20 weight %, based on the total weight of the oral care composition.

In at least one implementation, the oral care composition is free or substantially free of menthol, betaine, fluoride containing components, or a combination thereof.

In at least one implementation, the oral care composition may further include one or more flavorants, sweeteners, or combinations thereof.

In at least one implementation, the sweeteners may include one or more of isomalt, maltitol, xylitol, sorbitol, sucralose, or a combination thereof.

In at least one implementation, the oral care composition may further include one or more anticalculus agents. The anticalculus agents may include one or more phosphates, polyphosphates, or a combination thereof. The anticalculus agents may include one or more of tetrasodium pyrophosphate (TSPP), sodium tripolyphosphate (STPP), tetrapotassium pyrophosphate (TKPP), or a combination thereof.

In at least one implementation, the solid film may include a plurality of layers, optionally, a first layer of the plurality of layers may include the orally acceptable vehicle, further optionally, a second layer of the plurality of layers may include the one or more whitening agents.

In at least one implementation, the oral care composition may further include a third layer interposed between the first layer and the second layer, wherein the third layer may be an adhesive layer.

In at least one implementation, the second layer may be disposed adjacent the first layer.

In at least one implementation, the oral care composition may further include an abrasive, optionally, the abrasive comprises an insoluble phosphate salt, further optionally, the abrasive is present in an amount of from about 0.1 wt % to about 20 wt %, based on the total weight of the oral care composition.

The foregoing and/or other aspects and utilities embodied in the present disclosure may be achieved by providing a method for preparing any of the oral care compositions disclosed herein. The method may include contacting the orally acceptable vehicle, the one or more whitening agents, and water with one another to prepare a slurry. The method may also include preparing the solid film with the slurry.

In at least one implementation, preparing the solid film with the slurry may include casting at a temperature of from about 25° C. to about 80° C. or less, 75° C. or less, 70° C. or less, 65° C. or less, 60° C. or less, 50° C. or less, or 40° C. or less.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

Compositions disclosed herein may be or include an oral care product or an oral care composition (e.g., oral cleansing and whitening composition) thereof. For example, the composition may be an oral care product including the oral care composition and/or one or more additional ingredients/components. In another example, the composition may be the oral care composition of the oral care product. As used herein, the expression “oral care product” may refer to the final form which is sold to a consumer or administered to a user (e.g., patient). The oral care product may be a product that includes one or more chemical compounds or chemical compositions that may be applied to an oral cavity or a surface (e.g., enamel) thereof to therapeutically or non-therapeutically treat a condition (e.g., whiten teeth), deliver a benefit agent, improve the health of the user's oral cavity (e.g., vestibule, lips, jaws, palate, teeth, tongue, etc.), or a combination thereof.

The oral care product or the oral care composition thereof may include a carrier or an orally acceptable vehicle and one or more whitening agents. For example, the one or more whitening agents may be combined with, dispersed in, mixed with, or otherwise contacted with the carrier or the orally acceptable vehicle. It should be appreciated that the whitening agents may generally be unstable and/or may degrade in conventional oral care compositions or conventional oral care carriers. As further described herein, the carrier or the orally acceptable vehicle disclosed herein may be capable of or configured to prevent, reduce, or otherwise inhibit the degradation and/or improve the stability of the whitening agents.

The oral care product or the oral care composition thereof may be a solid. Illustrative oral care products or compositions of the present disclosure may be or include, but are not limited to, a toothpaste (dentifrice), an oral cleansing and whitening composition, a denture cleaner, or any other oral care product intended to contact the oral cavity and/or one or more surfaces of the oral cavity. In an exemplary implementation, the oral care product is an oral cleansing and whitening composition, similar to toothpaste, however, in the form of a solid film, ribbon, tape, strip, or the like. For simplicity, the term “toothpaste” is used herein to refers to the oral cleansing and whitening composition. The term “toothpaste” as used herein, however, does not limit the oral cleansing and whitening composition to a “paste,” but is simply used to refer to the conventional uses for a “toothpaste” and encompasses the conventional uses of toothpaste.

The oral care product or the oral care composition thereof may be a toothpaste in the form of a ribbon, strip, or tape. Each of the ribbons, strips, or tape, may be packaged as a single dose (e.g., single use strip) or a multi-dose (e.g., roll, stack, etc.) that may be separated by the user/consumer. The oral care product or the oral care composition thereof may be disposed of or packaged in a container, such as an airtight container. Illustrative containers may include, but are not limited to, a retort pouch, a sachet, a TETRA PAK®, a bottle, a tray, a metal can (e.g., tin can), or the like, or combinations thereof. In a preferred implementation, the oral care product or the oral care composition thereof may be disposed in a retort pouch or a sachet that is portable and/or disposable. It should be appreciated that each of the containers may include any number of servings or doses of the oral care product or the oral care composition thereof. For example, each container may include a single dose or multiple doses.

As noted above, the oral care product or the oral care composition thereof may be a toothpaste in the form of a ribbon, strip, or tape, such as a solid film. The solid film may be dissolvable and/or disintegrable. As used herein, the term or expression “dissolvable” may refer to the ability to disperse into a liquid. For example, a dissolvable solid film may disperse into a liquid, such as saliva. As used herein, the term or expression “disintegrable” may refer to the ability to decompose into constituent elements, parts, and/or small particles. It should be appreciated that the ability of the solid film to be dissolvable and/or disintegrable may be provided by one or more components of the oral cleansing and whitening composition. For example, the orally acceptable vehicle and/or the polymer base thereof may at least partially contribute to the dissolvable and/or disintegrable properties of the oral cleansing and whitening composition. In at least one implementation, the solid film may be dissolvable and/or disintegrable in water, saliva, or a combination thereof. The solid film may be dissolvable and/or disintegrable in a predetermined amount of time and/or rate. For example, the solid film may be completely dissolvable and/or disintegrable in about 30 seconds or less, about 1 min or less, about 2 min or less, about 3 min or less, about 5 min or less, about 10 min or less. In another example, the solid film may dissolve or disintegrate at a rate of about 0.5 gram/minute (g/min) or less, about 1 g/min or less, about 1.5 g/min or less, about 2 g/min or less, about 3 g/min or less, about 5 g/min or less, about 10 g/min or less, about 15 g/min or less, or about 30 g/min or less. It should be appreciated that conventional oral care products or oral care compositions thereof in the form of a solid film are often not dissolvable or disintegrable; and thus, need to be physically removed (e.g., peeling, abrading, etc.) after a predetermined period of time.

The oral care product or the oral care composition thereof may be or include a solid film having a single layer or a plurality of layers. For example, the solid film may include the components/ingredients disclosed herein in a single layer, such as a single, monolithic, uniformly distributed layer. In another example, the solid film may include a first or base layer, a second layer disposed adjacent the first layer, a third layer disposed adjacent the second layer, and/or additional subsequent layers. It should be appreciated that each of the layers of the oral care product or the oral care composition thereof may include any one or more of the components disclosed herein. For example, the first layer of the oral care product or the oral care composition may include the orally acceptable vehicle or one or more components thereof, and a second layer may include the whitening agent. In an exemplary implementation, the first layer includes the orally acceptable vehicle, which may include one or more of a polymer base including one or more water soluble polymers, plasticizers and/or block co-polymers, surfactants, flavorants, additional ingredients, or a combination thereof, and the second layer may include one or more of the whitening agents. In another exemplary implementation, the first layer includes the orally acceptable vehicle, which may include one or more of a polymer base including one or more water soluble polymers, plasticizers and/or block co-polymers, surfactants, flavorants, additional ingredients, or a combination thereof, the second layer may include an adhesive layer, which may be or include any one or more of the block co-polymers of ethylene oxide and propylene oxide disclosed herein, and the third layer may include the whitening agents.

The oral cleansing and whitening composition may be anhydrous or non-aqueous prior to use. For example, the oral cleansing and whitening composition may be free of water or substantially free of water. As used herein, “free of water” or “substantially free of water” may refer to a composition that contains water in an amount of less than 5 weight %, less than 3 weight %, less than 1 weight %, less than 0.1 weight %, less than 0.05 weight %, less than 0.01 weight %, less than 0.005 weight %, or less than 0.0001 weight %, based on a total weight of the oral cleansing and whitening composition. In another implementation, the oral care product or the whitening composition thereof prior to use may have “low water content”. As used herein, “low water content” may refer to a composition that contains water in an amount greater than about 5 weight % or greater than about 10 weight %, and less than about 20 weight %, less than about 15 weight %, or less than about 10 weight %, based on a total weight of the oral cleansing and whitening composition.

In at least one implementation, contacting the oral cleansing and whitening composition with water may initiate a whitening effect. For example, contacting the whitening agents with water may initiate the release of hydrogen peroxide. As noted above, the oral cleansing and whitening composition may be in the form of a solid film or ribbon. As such, the water may be provided by saliva when contacting the solid film with surfaces of the oral cavity. The water may also be provided by water utilized in the cleaning of the oral cavity (e.g., during brushing). For example, the oral cleansing and whitening composition may be utilized in a manner similar to toothpaste, and added water may initiate a whitening effect. Similarly, the action of brushing or agitation with a toothbrush may facilitate the initiation of the whitening effect.

The one or more whitening agents may be or include materials or substances that are effective to, capable of, or configured to provide whitening of a tooth surface to which it is applied. The one or more whitening agents may be or include, but are not limited to, one or more sources of hydrogen peroxide, one or more non-peroxide bleaching agents, or a combination thereof. For example, the one or more whitening agents may include one or more sources of hydrogen peroxide. In another example, the one or more whitening agents may include one or more non-peroxide bleaching agents. In yet another example, the one or more whitening agents may include a combination of the source of hydrogen peroxide and the non-peroxide bleaching agents. The combination of the source of hydrogen peroxide and the non-peroxide bleaching agents may interact synergistically to enhance the whitening efficacy of the oral cleansing and whitening composition. For example, the combination of the source of hydrogen peroxide and the non-peroxide bleaching agents may provide more than additive whitening efficacy.

The one or more sources of hydrogen peroxide may be or include any compound or material capable of or configured to provide or release hydrogen peroxide to increase the whitening efficacy of the oral cleansing and whitening composition. The sources of hydrogen peroxide may be capable of or configured to release hydrogen peroxide when contacted with water (e.g., saliva). Illustrative sources of hydrogen peroxide may be or include, but are not limited to, hydrogen peroxide, organic peroxy compounds, calcium peroxide, a cross-linked polyvinylpyrrolidone (PVP) hydrogen peroxide complex, a polyvinylpyrrolidone (PVP) hydrogen peroxide complex, a hydrogen peroxide—silica complex, a percarbonate, such as sodium percarbonate, peroxy acid and/or salts thereof, PEROXYDONE™ XL 10F complex, which is commercially available from Ashland Inc. of Covington, KY, or the like, and/or a combination thereof. Peroxy acids and/or the salts thereof may include organic peroxy acids, such as alkyl peroxy acids, monoperoxyphthalate, mixtures thereof, as well as inorganic peroxy acid salts, such as persulfate, dipersulfate, percarbonate, perphosphate, perborate, or persilicate salts of alkali and alkaline earth metals, such as lithium, potassium, sodium, magnesium, calcium and barium, and/or a combination thereof. Illustrative organic peroxy compounds may be or include, but are not limited to, carbamide peroxide or urea hydrogen peroxide, glyceryl hydrogen peroxide, alkyl hydrogen peroxides, dialkyl peroxides, alkyl peroxy acids, peroxy esters, diacyl peroxides, benzoyl peroxide, monoperoxyphthalate, and/or a combination thereof. The source of the hydrogen peroxide includes a complex of hydrogen peroxide adsorbed into a cross-linked polyvinylpyrrolidone (PVP). In an exemplary implementation, the source of hydrogen peroxide includes a hydrogen peroxide solution, such as a 50% hydrogen peroxide solution.

The amount or concentration of the source of hydrogen peroxide may vary widely, and may at least partially depend upon the amount or a desired amount of hydrogen peroxide provided or otherwise delivered by the source of hydrogen peroxide. In at least one implementation, the source of hydrogen peroxide may be present in an amount sufficient to provide from greater than 0 weight % or greater than or equal to about 0.01 weight % to less than or equal to about 3 weight % free hydrogen peroxide, less than or equal to about 10 weight % free hydrogen peroxide, or less than or equal to about 25 weight % free hydrogen peroxide, based on the total weight of the oral cleansing and whitening composition. For example, the source of hydrogen peroxide may be present in an amount sufficient to provide hydrogen peroxide (e.g., free hydrogen peroxide) in an amount of from greater than 0 weight %, about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, or about 1.0 weight % to about 1.1 weight %, about 1.2 weight %, about 1.3 weight %, about 1.4 weight %, about 1.5 weight %, about 1.6 weight %, about 1.7 weight %, about 1.8 weight %, about 1.9 weight %, about 2.0 weight %, about 3.0 weight %, about 4.0 weight %, about 5.0 weight %, about 8.0 weight %, or about 10.0 weight %. In another example, the source of hydrogen peroxide may be present in an amount sufficient to provide hydrogen peroxide in an amount of from greater than 0 weight % to about 3.0 weight %, about 0.1 weight % to about 2.0 weight %, about 0.2 weight % to about 1.9 weight %, about 0.3 weight % to about 1.8 weight %, about 0.4 weight % to about 1.7 weight %, about 0.5 weight % to about 1.6 weight %, about 0.6 weight % to about 1.5 weight %, about 0.7 weight % to about 1.4 weight %, about 0.8 weight % to about 1.3 weight %, about 0.9 weight % to about 1.2 weight %, or about 1.0 weight % to about 1.1 weight %. In yet another example, the source of hydrogen peroxide may be present in an amount sufficient to provide hydrogen peroxide in an amount greater than 0 weight % and less than or equal to 3.0 weight %, less than or equal to 2.0 weight %, less than or equal to 1.8 weight %, less than or equal to 1.6 weight %, less than or equal to 1.4 weight %, less than or equal to 1.2 weight %, less than or equal to 1.0 weight %, less than or equal to 0.8 weight %, less than or equal to 0.6 weight %, or less than or equal to 0.4 weight %. In an exemplary implementation, the source of hydrogen peroxide may be present in an amount sufficient to provide hydrogen peroxide in an amount of from about 1.5 weight % to about 3.0 weight %, based on the total weight of the oral cleansing and whitening composition.

The one or more non-peroxide bleaching agents may be or include, but are not limited to, a salt of a peroxymonosulfate or a salt of monopersulfate (MPS). For example, the non-peroxide bleaching agent may be or include, but is not limited to, an alkali metal salt of MPS, such as a potassium MPS, sodium MPS, ammonium MPS, or the like, or any combination thereof. MPS may be provided as a single molecule, a compound, such as a monopersulfate compound or MPS compound, a complex, such as a monopersulfate complex, or any combination thereof. In at least one implementation, the non-peroxide bleaching agents may be or include an MPS compound or a mixture of two or more salts of peroxymonosulfate. The MPS compound may be or include a mixed salt or triple salt, such as 2KHSO₅·KHSO₄·K₂SO₄. The MPS may be utilized as a solid, such as a solid powder. The MPS may also be utilized as a liquid, such as an aqueous liquid, that may be subsequently dried.

In at least one implementation, the non-peroxide bleaching agent may be or include OXONE®, which is commercially available from DuPont of Wilmington, DE. It should be appreciated that OXONE® has an active oxygen content of about 4.5%. The active oxygen content of the mixed salt is about 5.2% when the salt is purified. The active oxygen content of KHSO₅ is about 10.5%. It should further be appreciated that the pure mixed salt has about half as much active oxygen as compared to the pure form, and the 86.5% pure mixed salt (i.e., OXONE®) has 43% as much active oxygen as compared to the pure form. It should also be appreciated that OXONE® may include about 43% potassium hydrogen peroxymonosulfate, about 23% potassium hydrogen sulfate, about 29% potassium sulfate, about 3% potassium peroxydisulfate, and about 2% magnesium carbonate. MPS is also commercially available as CAROAT® from United Initiators Inc. of Mason, OH.

In at least one implementation, the non-peroxide bleaching agent may be provided as a solid. For example, the non-peroxide bleaching agent may be provided as a powder, a tablet, granules, or the like, and the non-peroxide bleaching agent may be combined with the remaining components to form the oral care product (e.g., solid film) or the oral cleansing and whitening composition thereof. It should be appreciated that the solid may be free or substantially free of water. The solid may be provided in a variety of forms, including, but not limited to, a free flowing granulation, a tablet (e.g., effervescing tablet), a caplet, granules, pellets, wafers, films, beads, or the like.

The amount or concentration of the non-peroxide bleaching agent may vary widely. In at least one implementation, the non-peroxide bleaching agent may be provided in an amount of from greater than 0.0 weight % to about 15 weight %, based on the total weight of the oral care product or the oral care composition thereof. For example, the non-peroxide bleaching agent may be provided in an amount from greater than 0.0 weight %, about 0.5 weight %, about 1 weight %, about 1.5 weight %, or about 2 weight % to about 2.5 weight %, about 3 weight %, about 3.5 weight %, about 4 weight %, about 4.5 weight %, about 5 weight %, about 10 weight %, or about 15 weight %, based on the total weight of the oral care product or the oral cleansing and whitening composition thereof. In another example, the non-peroxide bleaching agent may be provided in an amount from greater than 0.0 weight % to about 5 weight %, about 1 weight % to about 4 weight %, or about 2 weight % to about 3 weight %, based on the total weight of the oral care product or the oral cleansing and whitening composition thereof.

The orally acceptable vehicle may be or include a polymer base. The polymer base may be or include one or more water soluble polymers. The water soluble polymers may be capable of or configured to form a polymer matrix. Illustrative water soluble polymers may be or include, but are not limited to, one or more of carboxymethyl cellulose (CMC), hydroxyethyl cellulose (HEC), cetyl hydroxyethylcellulose, hydroxypropyl methylcellulose (HPMC), hydroxypropyl cellulose (HPC), polyvinylpyrrolidone (PVP), polyvinylpyrrolidone/vinyl acetate (PVP/VA), cellulose ethers, or the like, or combinations thereof. Illustrative cellulose ethers may be or include, but are not limited to, hydroxyethyl cellulose (HEC), hydroxypropyl cellulose (HPC), water soluble ethylhydroxyethyl cellulose (EHEC), carboxymethyl cellulose (CMC), carboxymethylhydroxyethyl cellulose (CMHEC), hydroxypropylhydroxyethyl cellulose (HPHEC), methyl cellulose (MC), methylhydroxypropyl cellulose (MHPC), methylhydroxyethyl cellulose (MHEC), carboxymethylmethyl cellulose (CMMC), hydrophobically modified carboxymethyl cellulose (HMCMC), hydrophobically modified hydroxyethyl cellulose (HMHEC), hydrophobically modified hydroxypropyl cellulose (HMHPC), hydrophobically modified ethylhydroxyethyl cellulose (HMEHEC), hydrophobically modified carboxymethylhydroxyethyl cellulose (HMCMHEC), hydrophobically modified hydroxypropylhydroxyethyl cellulose (HMHPHEC), hydrophobically modified methyl cellulose (HMMC), hydrophobically modified methylhydroxypropyl cellulose (HMMHPC), hydrophobically modified methylhydroxyethyl cellulose (HMMHEC), hydrophobically modified carboxymethylmethyl cellulose (HMCMMC), cationic hydroxyethyl cellulose (cationic HEC) and cationic hydrophobically modified hydroxyethyl cellulose (cationic HMHEC), a 2-pyrrolidinone,1 ethenyl-, homopolymer (CAS No. 9003-39-8), FLEXITHIX™, which is commercially available from Ashland of Kennedy, TX, an ammonium acryloyldimethyltaurate/vinylpyrrolidone copolymer, or copolymer of ammonium acryloyldimethyltaurate and vinylpyrrolidone monomers (CAS No. 91081-14-0), polyacrylate crosspolymer-6 (CAS No. 1439374-06-7), or the like, or combinations thereof.

In an exemplary implementation, the water-soluble polymers include polyvinylpyrrolidone (PVP). The PVP may be low molecular weight PVP, high molecular weight PVP, or a combination thereof. As used herein, “low molecular weight PVP” may refer to PVP having a molecular weight of from about 10,000 Da or less to about 100,000 Da. As used herein, “high molecular weight PVP” may refer to PVP having a molecular weight of from about 200,000 Da to about 1,500,000 Da. As further demonstrated herein, the combination of high and low molecular weight PVP facilitates the stability of the one or more whitening agents. Also, as further demonstrated herein, the combination of the high and low molecular may prevent, reduce, or inhibit to degradation of the one or more whitening agents.

The polymer base may be present in an amount of from about 5 weight % to about 40 weight %, based on the total weight of the oral care product or the oral cleansing and whitening composition thereof. For example, the polymer base may be present in an amount of from about 5 weight %, about 10 weight %, about 15 weight %, or about 20 weight % to about 25 weight %, about 30 weight %, about 35 weight %, or about 40 weight %, based on the total weight of the oral care product or the oral cleansing and whitening composition thereof. As noted above, the polymer base may include a combination of high molecular weight PVP and low molecular weight PVP. The high molecular weight PVP and the low molecular weight PVP may be present in a weight % ratio of from about 0.5:1, about 0.6:1, about 0.7:1, about 0.8:1, or about 0.9:1 to about 1:1, about 1.1:1, about 1.2:1, about 1.3:1, about 1.4:1, about 1.5:1, about 1.6:1, about 1.7:1, about 1.8:1, about 1.9:1, or about 2:1. In another implementation, the polymer base may include only the high molecular weight PVP or the low molecular weight PVP.

The oral care product, oral cleansing and whitening composition, or the carrier thereof may be substantially clear. As used herein, the expression “substantially clear” may refer to the oral care product, the oral cleansing and whitening composition, or the carrier that is translucent or transparent.

In at least one implementation, the orally acceptable vehicle may include one or more plasticizers and/or polymers capable of or configured to improve flexibility of the oral care product (e.g., solid film oral cleansing and whitening composition). Illustrative plasticizers may be or include, but are not limited to, glycerin, sorbitol, an alkylene glycol, poloxamers, block co-polymers, or combinations thereof. Illustrative alkylene glycol may be or include, but are not limited to polyethylene glycol (PEG) or propylene glycol (PPG). In an exemplary implementation, the plasticizer may include polyethylene glycol, propylene glycol, or a combination thereof. Illustrative polyethylene glycol may be or include, but are not limited to, PEG-400, PEG-600, PEG-1000, or a combination thereof. The poloxamers may be a liquid or a paste. The poloxamer may have an average molecular weight of less than or equal to about 12,000 Dalton (Da), less than or equal to about 11,000 Da, less than or equal to about 10,000 Da, less than or equal to about 9,000 Da, less than or equal to about 8,000 Da, less than or equal to about 7,000 Da, or less than or equal to about 6,000 Da. Illustrative poloxamers may be or include, but are not limited to, one or more of PLURONIC® L35 (poly(ethylene glycol)-block-poly(propylene glycol)-block-poly(ethylene glycol)), PLURONIC® L43, PLURONIC® L64, PLURONIC® L10, PLURONIC® L44, PLURONIC® L62, PLURONIC® 10R5, PLURONIC® 17R4, PLURONIC® L25R4, PLURONIC® P84, PLURONIC® P65, PLURONIC® P104, and PLURONIC® P105, or the like, or any mixture or combination thereof, each of which or commercially available from BASF Corp. of Florham Park, NJ. In a preferred implementation, the orally acceptable vehicle includes a poly(ethylene glycol)-block-poly(propylene glycol)-block-poly(ethylene glycol) polymer, such as PLURONIC® L35. The block co-polymers may be or include one or more block co-polymers of polyethylene glycol and polyethylene glycol (e.g., molecular weight of at least 5000 Da), polypropylene glycol and polyethylene glycol, and the like, and combinations thereof. The block co-polymer of ethylene oxide and propylene oxide may be represented by the formula (1),

(ethylene oxide)ₓ−(propylene oxide)_{y} (1)

where x is an integer from about 80 to about 150 (e.g., x=100-130, or about 116 or about 118), and y is an integer from about 30 to about 80 (e.g., y=60-70, or about 66). The block co-polymer of ethylene oxide and propylene oxide may have an average molecular weight greater than or equal to about 5,000 Da and less than or equal to about 20,000 Da. For example, the molecular weight of the block co-polymer of ethylene oxide and propylene oxide may be from about 8,000 Da to about 13,000 Da. In another example, the molecular weight of the block co-polymer of ethylene oxide and propylene oxide may be from about 9,800 Da or about 10,000 Da. In yet another example, the molecular weight of the block co-polymer of ethylene oxide and propylene oxide may be from about 8,000 Da to about 10,000 Da. In at least one implementation, the block copolymer, if included in the composition, may be or include PLURAFLO® L4370, PLURAFLO® L1220, and PLURACARE® L1220, each of which are commercially available from BASF of Wyandotte, MI.

The one or more plasticizers may be present in an amount of from about 1 weight % to about 40 weight %, based on the total weight of the oral care product or the oral cleansing and whitening composition thereof. For example, the one or more plasticizers may be present in an amount of from about 1 wt %, about 5 wt %, or about 10 wt % to about 15 wt %, about 20 wt %, about 30 wt %, or about 40 wt %, based on the total weight of the oral care product or the oral cleansing and whitening composition thereof. In at least one implementation, the plasticizers include propylene glycol in an amount greater than 6 weight %, greater than 7 weight %, greater than 8 weight %, or greater than 9 weight %, based on the total weight of the oral care product or the oral care composition thereof. As demonstrated herein, providing the propylene glycol in an amount greater than 6 wt % resulted in a corresponding increase in flexibility and transparency of the prepared solid films. The plasticizers may also include polyethylene glycol, such as PEG-600, in lieu of or in addition to the propylene glycol. The PEG or PEG-600 may be present in an amount of about 4 wt % to about 20 wt %, about 8 wt % to about 16 wt %, about 10 wt % to about 14 wt %, or about 12 wt %, based on the total weight of the oral care product or the oral care composition thereof. As demonstrated herein, the inclusion of PEG or PEG-600 resulted in improved stability and/or reduced degradation of the whitening agents. The PEG or PEG-600 further improved the flexibility of the solid films.

The oral cleansing and whitening composition or the carrier thereof may include one or more surfactants. For example, the oral cleansing and whitening composition may include one or more anionic surfactants, one or more cationic surfactants, one or more zwitterionic surfactants, one or more nonionic surfactants, or combinations thereof. Examples of suitable surfactants may be found in U.S. Pat. No. 3,959,458 to Agricola et al., U.S. Pat. No. 3,937,807 to Haefele, and U.S. Pat. No. 4,051,234 to Gieske et al., the disclosures of which are incorporated herein by reference in their entirety to the extent they are consistent with the present disclosure.

Illustrative anionic surfactants may include, but are not limited to, water-soluble salts of higher fatty acid monoglyceride monosulfates, such as a sodium salt of a monosulfated monoglyceride of hydrogenated coconut oil fatty acids, such as sodium N-methyl N-cocoyl taurate, sodium cocomonoglyceride sulfate. Illustrative anionic surfactants may also include higher alkyl sulfates. As used herein, “higher alkyl” refers to C₆₋₃₀ alkyl. For example, in a preferred implementation the anionic surfactant is sodium lauryl sulfate. The anionic surfactants may also include higher alkyl-ether sulfates. For example, the anionic surfactants may have a formula CH₃(CH₂)ₘCH₂(OCH₂CH₂)ₘOSO₃X, where m is 6-16, n is 1-6, and X is Na or K. In an exemplary implementation, m is 10, and n is 2, 3, or 4, and X is Na or K. For example, the anionic surfactant may be sodium laureth-2 sulfate (CH₃(CH₂)₁₀CH₂(OCH₂CH₂)₂OSO₃Na). In another implementation, the anionic surfactant may include higher alkyl aryl sulfonates, such as sodium dodecyl benzene sulfonate (sodium lauryl benzene sulfonate), and higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. In an exemplary implementation, the anionic surfactant is a water soluble salt of alkyl sulfates having from 10 to 18 carbon atoms in the alkyl radical and water soluble salts of sulfonated monoglycerides of fatty acids having from 10 to 18 carbon atoms. For example, the anionic surfactant may be or include, sodium lauryl sulfate, sodium lauroyl sarcosinate, sodium coconut monoglyceride sulfonates, or the like, and mixtures thereof. In an exemplary implementation, the oral cleansing and whitening composition includes sodium lauryl sulfate (SLS).

The nonionic surfactant may function as an emulsifier. Illustrative nonionic surfactants may be or include, but are not limited to, poloxamers or the like. For example, the nonionic surfactants may include polysorbate 20, poloxamer 407, poloxamer 338, or the like, and mixtures thereof. The nonionic surfactants may also include, but are not limited to, ethoxylated and hydrogenated ethoxylated castor oils, such as those commonly designated as PEG NN castor oil or PEG NN hydrogenated castor oil, where “NN” designates the number of ethylene oxide units polymerized onto the castor oil to form the nonionic surfactant. For example, the nonionic surfactants may be or include PEG 16, 20, 25, 30, 40, 50, 60, 80, 100, 200, 300, 400, 500, 600, or a combination thereof. In a preferred implementation, the nonionic surfactant is polysorbate 20.

The amphoteric and zwitterionic surfactants may be or include, but are not limited to, derivatives of C₈₋₂₀ aliphatic secondary and tertiary amines having an anionic group such as carboxylate, sulfate, sulfonate, phosphate or phosphonate. Illustrative amphoteric and zwitterionic surfactants may include, but are not limited to, sultaines and betaines, such as cocamidopropyl betaine (CAPB), derivatives of aliphatic secondary and tertiary amines in which the aliphatic radical can be a straight or branched chain and wherein one of the aliphatic substituents contains about 8-18 carbon atoms and one contains an anionic water-solubilizing group, such as carboxylate, sulfonate, sulfate, phosphate or phosphonate, or the like, or a combination thereof. In an exemplary implementation, the oral care product, the oral cleansing and whitening composition, or the carrier thereof may include one or more betaines. The one or more betaines may be capable of or configured to facilitate the release of the solid film from a substrate, for example, during manufacture or use. For example, the one or more betaines may be utilized as a releasing agent. The betaines may be utilized as an about 30% betaines solution. It should be appreciated that other surfactants may also be utilized in combination with or in lieu of the betaines. For example, an SLS solution may also be utilized as the releasing agent.

The amount of any one or more of the surfactants present in the oral care composition or carrier thereof may vary widely. In at least one implementation, the amount of any one or more of the surfactants present in the oral care composition or the carrier thereof may be greater than 0.0 weight % or 0.1 weight % and less than or equal to 10.0 weight %, based on a total weight of the oral care composition or the component thereof. For example, the amount of any one or more of the surfactants present in the oral care composition or the carrier thereof may be from greater than 0 weight %, about 0.1 weight %, about 0.5 weight %, about 1 weight %, about 1.5 weight %, about 2 weight %, or about 2.5 weight % to about 3 weight %, about 3.5 weight %, about 4 weight %, about 4.5 weight %, about 5 weight %, about 8 weight %, or about 10 weight %, based on a total weight of the oral care composition or the carrier thereof. In another example, the amount of any one or more of the surfactants present in the oral care composition or the carrier thereof may be from about 0.5 weight % to about 5 weight %, about 1 weight % to about 4.5 weight %, about 1.5 weight % to about 4 weight %, about 2 weight % to about 3.5 weight %, or about 2.5 weight % to about 3 weight %, based on a total weight of the oral care composition or the carrier thereof. In an exemplary implementation, each of the one or more of the surfactants may, separately and independently, be present in the oral care composition or the carrier thereof in an amount of from about 0.01 weight % to about 3 weight %, about 1 weight % to about 2 weight %, about 1.25 weight % to about 1.75 weight %, about 1.5 weight %, about 1 weight %, or about 0.5 weight %, based on a total weight of the oral care composition or the carrier thereof.

The oral care product or the oral care composition thereof may also include one or more flavorants and/or sweeteners. Illustrative flavorants and/or sweeteners may include, but are not limited to, essential oils and various flavoring aldehydes, esters, alcohols, or the like. The flavorants and/or sweeteners may also include, but are not limited to, sweeteners, sucralose, maltodextrin, dextrose, polydextrose, sucrose, maltose, dextrin, dried invert sugar, mannose, xylose, ribose, fructose, levulose, galactose, sugar alcohols, such as sorbitol, mannitol, xylitol, maltitol, and isomalt, aspartame, neotame, saccharin and salts thereof (e.g., sodium saccharin), gum arabic, dipeptide-based intense sweeteners, cyclamates, dihydrochalcones, or the like, or mixtures thereof. Examples of the essential oils include oils of spearmint, peppermint, wintergreen, sassafras, clove, sage, eucalyptus, marjoram, cinnamon, lemon, lime, grapefruit, and orange. In another example, the flavoring agents may include menthol, carvone, and anethole. In a preferred implementation, the oral care product or the oral care composition thereof is a sugarless composition to prevent or inhibit tooth decay. For example, the sweeteners may include one or more of isomalt, maltitol, xylitol, sorbitol, sucralose, or any combination thereof. In an exemplary implementation, the oral care product or the oral care composition thereof is free or substantially free of menthol. As further described herein, the inclusion of menthol may facilitate the degradation of one or more of the whitening agents.

It should be appreciated by one having ordinary skill in the art that the oral care products and/or the oral care composition thereof may include other additional ingredients/components. For example, the oral care products and/or the oral care composition thereof may include any one or more of the following: anti-caries agents, anticalculus agents, diluents, surface active agents or surfactants, mouth feel agents, sweetening agents, colorants or coloring agents, preservatives, antifoam agents (e.g., benzoic acid, sulfuric acid, glyceryl monostearate, etc.), abrasives, or the like, or a combination thereof. It should further be appreciated by one having ordinary skill in the art that while general attributes of each of the above categories of materials may differ, there may be some common attributes and any given material may serve multiple purposes within two or more of such categories of materials. For example, clove oil may be capable of or configured to provide a flavor property as well as a therapeutic function. In at least one implementation, the oral care products and/or the oral care composition thereof may be free or substantially free of fluoride containing components.

In an exemplary implementation, the oral care product or the oral care composition thereof may include one or more anticalculus agents. Illustrative anticalculus agents may include, but are not limited to, phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates. In a typical implementation, the anticalculus agents includes tetrasodium pyrophosphate (TSPP), sodium tripolyphosphate (STPP), tetrapotassium pyrophosphate (TKPP), or a combination thereof.

In at least one implementation, the additional components of the oral care product or the oral care composition thereof may include one or more abrasives or abrasive compounds. As used herein, the term “abrasive” may refer to materials commonly referred to as “polishing agents.” The one or more abrasives may be mixed, combined, dispersed, suspended, or otherwise contacted with the orally acceptable vehicle. In at least one implementation, the oral care product or the oral care composition thereof includes a single abrasive. In another implementation, the oral care product or the oral care composition thereof includes a mixture or combination of at least two abrasives. Illustrative abrasives may be or include, but are not limited to, metaphosphate compounds, phosphate salts (e.g., insoluble phosphate salts), such as sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate, calcium carbonate, magnesium carbonate, hydrated alumina, abrasive silicas, silica, silicates, zirconium silicate, aluminum silicate, calcined aluminum silicate, polymethyl methacrylate, or the like, or a combination thereof. In an exemplary implementation, the abrasives include at least the silica abrasives, such as those available under the trade designation ZEODENT® 115, which are commercially available from Evonic Industries, AG of Essen Germany. Any one or more of the abrasives may be present in an amount of from 0 wt % to about 20 wt %, based on the total weight of the oral care product or the oral care composition thereof. For example, the one or more abrasives may be present in an amount of from 0 wt %, about 0.1 wt % about 0.5 wt %, about 1 wt %, or about 2 wt % to less than or equal to about 5 wt %, less than or equal to about 8 wt %, less than or equal to about 10 wt %, less than or equal to about 15 wt %, or less than or equal to about 20 wt %, based on the total weight of the oral care product or the oral care composition thereof.

All ingredients for use in the compositions described herein should be orally acceptable. As used herein, “orally acceptable” may refer any ingredient that is present in a composition as described in an amount and form which does not render the composition unsafe for use in the oral cavity.

The present disclosure may provide a method for preparing the oral care product or an oral care composition thereof. The method may include preparing a slurry of the orally acceptable vehicle and the one or more whitening agents. The slurry may be prepared by mixing, stirring, combining, or otherwise contacting the orally acceptable vehicle, the one or more whitening agents, and water with one another. The water may be present in an amount sufficient to prepare a slurry. For example, the water may be present in an amount greater than or equal to about 40 wt %, greater than or equal to about 45%, greater than or equal to about 50%, greater than or equal to about 55%, greater than or equal to about 60%, greater than or equal to about 65%, greater than or equal to about 70%, or more. The method may also include preparing solid films of the oral care product with the slurry. Preparing the solid films of the oral care product with the slurry may include at least partially evaporating water from the slurry with heat and/or reduced pressure.

Preparing the solid films of the oral care product may include casting the slurry on a substrate (e.g., stainless steel surface) prior to evaporating the water therefrom. Casting may refer to the application or disposition of the slurry to the substrate at an appropriate, sufficient, or predetermined thickness. The substrate may be a metallic substrate, such as a stainless steel substrate, a non-stick substrate, such as a mylar or TEFLON surface, or the like. The casting may be conducted at temperatures of from room temperature or about 25° C. to about 60° C. or more, such as up to about 75° C., about 80° C., or more. As demonstrated herein, relatively lower temperatures are preferred when utilizing a peroxide source to prevent or inhibit the degradation of the whitening agents. The casting may be conducted for a predetermined amount of time (i.e., casting time), which may be at least partially determined by the amount of water present in the slurry. The casing time may be from about 1 hour (h), about 2 h, about 5 h, about 10 h, about 12 h, about 1 day (d), about 2 d, about 4 d, about 1 week, or more. As used herein, the expression “casting time” may refer to the amount of time for drying the slurry.

In at least one implementation, a releasing agent may be utilized to facilitate the formation of the solid films during casting. For example, the releasing agent (e.g., betaines) may be incorporated into the slurry or the oral care composition to facilitate the separation of the solid films from the substrate. In another example, the releasing agent may be disposed or coated on the substrate prior to casting the slurry on the substrate to facilitate the separation of the solid films from the substrate. In yet another example, the releasing agent may be disposed or coated on the substrate and incorporated into the slurry or the oral care composition. The amount of the releasing agent utilized may be limited. For example, the amount of the releasing agent may be limited to prevent the degradation or instability of the whitening agent.

The solid film oral cleansing and whitening compositions disclosed herein may retain active oxygen (AO) in an amount of at least 50%, at least 55%, at least 60%, at least 65%, at least 70%, at least 75%, at least 80%, at least 85%, or at least 90%. For example, the components of the oral care product or the oral cleansing and whitening compositions disclosed herein may be selected to reduce, prevent, or otherwise inhibit the loss of active oxygen (AO) during fabrication and/or after exposure to one or more aging conditions.

Active oxygen (AO %) may be determined according to Formulas (1)-(3):

$$AO \% in Film = \frac{Oxygen in Film}{Film Weight} \times 100 \qquad ( 1 )$$

$$Solid \% = \frac{Film weight}{Slurry Weight} \times 100 \qquad ( 2 )$$

$$AO \% in Film = \frac{Oxygen in Film}{Solid \% \times Slurry Weight} \times 100 = \frac{AO \% in Slurry}{Solid \%} \times 100. \qquad ( 3 )$$

The methods of preparing the solid film oral cleansing and whitening compositions disclosed herein may be capable of retaining active oxygen (AO) in an amount of at least 50%, at least 55%, at least 60%, at least 65%, at least 70%, at least 75%, at least 80%, at least 85%, or at least 90%. For example, the methods of preparing the solid film oral cleansing and whitening compositions may reduce, prevent, or otherwise inhibit the loss of active oxygen (AO) during fabrication and/or after exposure to one or more aging conditions.

In at least one implementation, the slurry may have a pH of from about 3.0 to about 5.0. For example, the slurry prior to casting may have a pH of from about 3.0, about 3.2, about 3.4, about 3.6, about 3.8, or about 4.0 to about 4.2, about 4.4, about 4.6, about 4.8, or about 5.0. The slurry may be adjusted to have a pH of from about 2.0 to about 8.0. Maintaining the pH below 7.0 or from about 3.0 to about 5.0 may improve stability of the slurry. For example, the slurry and/or one or more components therein may decompose at relatively a relatively higher pH. For example, the one or more whitening agents may decompose at an accelerated rate at a pH greater than about 4.0, greater than about 4.5, greater than about 5.0, or greater.

The oral care composition after casting and/or during use (e.g., as a solid film) as measured in saliva may have a pH of about 8 to about 10. For example, the oral care composition after casting and/or during use may have a pH of from about 8, about 8.2, about 8.4, about 8.6, about 8.8, or about 9 to about 9.2, about 9.4, about 9.6, about 9.8, or about 10. The relatively higher pH may be attributed to the use of whitening agent and/or peroxide compatible insoluble alkaline agents. For example, the relatively higher pH may be attributed to the use of the phosphates and/or polyphosphates, which are compatible with the whitening agents, such as the peroxides.

The following numbered paragraphs disclose one or more exemplary variations of the subject matter of the application:
1. An oral care composition, comprising an orally acceptable vehicle and one or more whitening agents, wherein the oral care composition is a toothpaste in the form of a solid film, and wherein the one or more whitening agents comprise at least one of a source of hydrogen peroxide, a non-peroxide bleaching agent, or a combination thereof, and wherein the solid film is dissolvable and/or disintegrable.
2. The oral care composition of paragraph 1, wherein the whitening agent comprises the source of hydrogen peroxide.
3. The oral care composition of paragraph 1 or 2, wherein the source of hydrogen peroxide comprises a hydrogen peroxide solution.
4. The oral care composition of paragraph 1 or 2, wherein the source of hydrogen peroxide is a solid, wherein the source of hydrogen peroxide comprises one or more of a cross-linked polyvinylpyrrolidone (PVP) hydrogen peroxide complex, a polyvinylpyrrolidone (PVP) hydrogen peroxide complex, carbamide peroxide, a percarbonate, a hydrogen peroxide—silica complex or a combination thereof.
5. The oral care composition of any of the foregoing paragraphs, wherein the source of hydrogen peroxide is present in an amount sufficient to provide free hydrogen peroxide in an amount of from about 0.01 weight % to less than or equal to about 10 weight %, based on the total weight of the oral care composition.
6. The oral care composition of any of the foregoing paragraphs, wherein the whitening agent comprises the non-peroxide bleaching agent.
7. The oral care composition of any of the foregoing paragraphs, wherein the non-peroxide bleaching agent comprises a salt of monopersulfate (MPS).
8. The oral care composition of any of the foregoing paragraphs, wherein the salt of MPS comprises one or more of potassium MPS, sodium MPS, ammonium MPS, or a combination thereof.
9. The oral care composition of paragraph 7, wherein the salt of MPS comprises 2KHSO₅·KHSO₄·K₂SO₄.
10. The oral care composition of any of the foregoing paragraphs, wherein the non-peroxide bleaching agent is present in an amount of from greater than 0 weight % to less than or equal to about 15 weight %.
11. The oral care composition of any of the foregoing paragraphs, wherein the orally acceptable vehicle comprises a polymer base comprising one or more water soluble polymers.
12. The oral care composition of paragraph 11, wherein the one or more water soluble polymers comprise one or more of carboxymethyl cellulose (CMC), hydroxyethyl cellulose (HEC), cetyl hydroxyethylcellulose, hydroxypropyl methylcellulose (HPMC), hydroxypropyl cellulose (HPC), polyvinylpyrrolidone (PVP), polyvinylpyrrolidone/vinyl acetate (PVP/VA), cellulose ethers, or a combination thereof.
13. The oral care composition of paragraph 11 or 12, wherein the one or more water soluble polymers comprise polyvinylpyrrolidone (PVP).
14. The oral care composition of paragraph 13, wherein the PVP comprises a combination of high molecular weight PVP and low molecular weight PVP.
15. The oral care composition of paragraph 14, wherein the high molecular weight PVP comprises a molecular weight of from about 200,000 Da to about 1,500,000 Da, or about 300,000 Da to about 1,300,000 Da, or from 500,000 Da to about 1,000,000 Da.
16. The oral care composition of paragraphs 14 or 15, wherein the low molecular weight PVP comprises a molecular weight of from about 10,000 Da to about 100,000 Da, about 30,000 Da to about 70,000 Da, or about 40,000 Da to about 60,000, Da.
17. The oral care composition of paragraphs 14 to 16, wherein a weight ratio of the high molecular weight PVP to the low molecular weight PVP may be from about 0.5:1 to about 2:1.
18. The oral care composition of any one of the foregoing paragraphs, further comprising one or more plasticizers.
19. The oral care composition of paragraph 18, wherein the plasticizer comprises one or more of glycerin, sorbitol, an alkylene glycol, or a combination thereof.
20. The oral care composition of paragraph 19, wherein the plasticizer comprises the alkylene glycol, wherein the alkylene glycol comprises one or more of polyethylene glycol, polypropylene glycol, or a combination thereof.
21. The oral care composition of paragraph 20, wherein the alkylene glycol comprises polyethylene glycol in an amount of from about 8 weight % to about 16 weight %, based on the total weight of the oral care composition.
22. The oral care composition of any one of the foregoing paragraphs, wherein the oral care composition is free or substantially free of menthol, betaine, fluoride containing components, or a combination thereof.
23. The oral care composition of any one of the foregoing paragraphs, further comprising one or more flavorants, sweeteners, or combinations thereof.
24. The oral care composition of paragraph 23, wherein the sweeteners comprises one or more of isomalt, maltitol, xylitol, sorbitol, sucralose, or a combination thereof.
25. The oral care composition of any of the foregoing paragraphs, further comprising one or more anticalculus agents, optionally, the anticalculus agents comprises one or more phosphates, polyphosphates, or a combination thereof, further optionally, the anticalculus agents comprises one or more of tetrasodium pyrophosphate (TSPP), sodium tripolyphosphate (STPP), tetrapotassium pyrophosphate (TKPP), or a combination thereof.
26. The oral care composition of any one of the foregoing paragraphs, wherein the solid film comprises a plurality of layers, optionally, a first layer of the plurality of layers comprises the orally acceptable vehicle, further optionally, a second layer of the plurality of layers comprises the one or more whitening agents.
27. The oral care composition of paragraph 26, further comprising a third layer interposed between the first layer and the second layer, wherein the third layer is an adhesive layer.
28. The oral care composition of paragraph 26, wherein the second layer is disposed adjacent the first layer.
29. The oral care composition of any one of the foregoing paragraphs, further comprising an abrasive, optionally, the abrasive comprises an insoluble phosphate salt, further optionally, the abrasive is present in an amount of from about 0.1 wt % to about 20 wt %, based on the total weight of the oral care composition.
30. A method for preparing the oral care composition of any one of the foregoing paragraphs, the method comprising: contacting the orally acceptable vehicle, the one or more whitening agents, and water with one another to prepare a slurry; and preparing the solid film with the slurry.
31. The method of paragraph 30, wherein preparing the solid film with the slurry comprises casting at a temperature of from about 25° C. to about 80° C. or less, 75° C. or less, 70° C. or less, 65° C. or less, 60° C. or less, 50° C. or less, or 40° C. or less.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

Exemplary oral care compositions (1)-(7) were prepared as solid films. To prepare the oral care compositions (1)-(7), respective slurries were prepared by combining the ingredients according to Table 1. Each of the slurries was then cast on a stainless steel surface with or without heat to prepare the solid films. The amount or yield of active oxygen (AO) from each of the solid films was evaluated under varying casting conditions, as summarized in Table 1.

TABLE 1
Oral Care Composition (1)-(7)

| Component | (1) | (2) | (3) | (4) | (5) | (6) | (7) |
|---|---|---|---|---|---|---|---|
| Water | 61.27 | 61.27 | 61.27 | 66.27 | 65.27 | 65.27 | 65.27 |
| Hydroxypropyl Cellulose (wt %) | 5 | — | 5 | — | — | — | — |
| High MW PVP (wt %) | 10 | 15 | 10 | 10 | 7.5 | 10 | 10 |
| Low MW PVP (wt %) | 10 | 10 | 10 | 10 | 7.5 | 10 | 10 |
| Propylene Glycol (wt %) | 4 | 4 | 4 | 4 | 4 | 4 | 4 |
| Sodium Lauryl Sulfate (wt %) | 1.2 | 1.2 | 1.2 | 1.2 | 1.2 | 1.2 | 1.2 |
| Betaine (wt %) | 2.2 | 2.2 | 2.2 | 2.2 | 2.2 | — | — |
| 50% HP Solution (wt %) | 3 | 3 | — | — | — | 3 | 3 |
| 3% PVP-H₂O₂ Solid (wt %) | — | — | 3 | 3 | 9 | — | — |
| Sucralose (wt %) | 0.4 | 0.4 | 0.4 | 0.4 | 0.4 | 0.4 | 0.4 |
| Menthol (wt %) | 0.08 | 0.08 | 0.08 | 0.08 | — | — | 0.08 |
| Excipient (wt %) | Bal. | Bal. | Bal. | Bal. | Bal. | Bal. | Bal. |
| Theoretical AO of Film (%) | 1.174 | 1.174 | 1.174 | 1.174 | 2.878 | 4.049 | 4.049 |
| Film overnight @ 100° C./0% RH (AO %) | — | 0.256 | — | — | — | — | — |
| Film about 1 wk @ 60° C./0% RH (AO %) | — | — | — | 0.3 | 2.28 | 3.07 | — |
| Film 5 days @ 60° C./0% RH (AO %) | — | — | — | — | — | 3.57 | — |
| Film overnight @ 60° C./0% RH (AO %) | 0.5649 | 0.056 | — | — | — | — | — |
| Film 3-4 hours @ 60° C./0% RH (AO %) | — | — | 1.088 | — | — | 2.74 | 3.59 |
| Film overnight @ 40° C./65% RH (AO %) | 0.9980 | — | 0.975 | — | — | — | — |
| Film 2 mo @ 40° C./65% RH (AO %)° | — | — | — | — | — | 3.21 | — |
| Film overnight @ 25° C. (AO %) | — | 0.9296 | 1.063 | — | — | — | — |

Active oxygen (AO %) was determined according to Formulas (1)-(3):

$$AO \% in Film = \frac{Oxygen in Film}{Film Weight} \times 100 \qquad ( 1 )$$

$$Solid \% = \frac{Film weight}{Slurry Weight} \times 100 \qquad ( 2 )$$

$$AO \% in Film = \frac{Oxygen in Film}{Solid \% \times Slurry Weight} \times 100 = \frac{AO \% in Slurry}{Solid \%} \times 100 \qquad ( 3 )$$

It should be appreciated that a relatively higher yield or recovery of active oxygen (AO) indicates increased stability of the whitening agent, such as the peroxide source in the solid film. Similarly, a relatively lower recover of AO indicates decreased stability of the whitening agent (e.g., the peroxide source) in the solid film. As indicated by oral care compositions (1) and (2), increasing casting temperatures resulted in decreased amounts of AO. Oral care composition (3) further supports the utilization of lower temperatures, as casting overnight at temperatures closer to room temperature or about 25° C. exhibited relatively higher AO as compared to 40° C., indicating improved stability.

As indicated in Table 1, aging the oral care composition (4) for about one week resulted in decreased AO recovery of about 0.3%. Without being bound by theory, it is believed that the significant decrease in the AO % resulted from interactions of the peroxide source with other components of the oral care composition. Particularly, it is believe that the interactions resulted in the decomposition of the peroxide source.

For the oral care composition (5), betaine was utilized in solution in the composition as well as a releasing agent to facilitate the formation or preparation of the solid film. As such, the relatively greater decomposition of the peroxide source was attributed to the increased amounts of betaine.

As indicated in Table 1, the oral care composition (6) did not include either betaine or menthol. The oral care composition (6) exhibited relatively greater stability of the peroxide source even after longer exposure at higher temperatures for a period of at least five days and at least two months. It should be appreciated that oral care compositions (6) and (7) were the same except for the addition of menthol. As illustrated in Table 1, the inclusion of menthol in the oral care composition (7) reduced the stability of the peroxide source. As such, without being bound by theory, it is believed that the menthol at least partially contributes to the decomposition of the peroxide source. It was also observed from the oral care compositions (1)-(7) that utilizing propylene glycol as a plasticizer resulted in improved flexibility.

### Example 2

Exemplary oral care compositions (8)-(12) were prepared as solid films. To prepare the oral care compositions (8)-(12), respective slurries were prepared by combining the ingredients according to Table 2. Each of the slurries was then cast under varying casting conditions, as indicated in Table 2.

TABLE 2
Oral Care Composition (8)-(12)

| Component | (8) | (9) | (10) | (11) |
|---|---|---|---|---|
| Water | 66.55 | 66.55 | 58.55 | 58.55 |
| Hydroxypropyl Cellulose (wt %) | — | — | — | — |
| High MW PVP (wt %) | 10 | 10 | 12 | 12 |
| Low MW PVP (wt %) | 10 | 10 | 12 | 12 |
| Propylene Glycol (wt %) | 6 | 6 | 10 | 10 |
| PEG 600 (wt %) | — | — | — | — |
| Sodium Lauryl Sulfate (wt %) | 1.2 | 1.2 | 1.2 | 1.2 |
| Betaine (wt %) | — | — | — | — |
| 50% HP Solution (wt %) | 3 | 3 | 3 | 3 |
| 3% PVP-H₂O₂ Solid (wt %) | — | — | — | — |
| Sucralose (wt %) | 0.4 | 0.4 | 0.4 | 0.4 |
| Menthol (wt %) | — | — | — | — |
| Excipient (wt %) | Bal. | Bal. | Bal. | Bal. |
| Temperature | 82.5 | 75 | 80 | 75 |
| Cast time (min) | 30 | 30 | 60 | 60 |
| Theoretical AO of Film (%) | 5.15 | 5.15 | 4.27 | 4.27 |
| Film initial AO (%) | — | 3.72 | 1.04 | 2.46 |
| Film 1 wk @ 60° C./0% RH (AO %) | — | — | — | 1.758 |

As indicated in oral care compositions (8)-(11), the amount of propylene glycol was increased relative to oral care compositions (1)-(7). It was discovered that increasing the relative amount of propylene glycol correspondingly increased the flexibility and transparency of the respective solid films. Increasing the relative amount of PVP also resulted in improved the ability to handle and removed the solid films from the casting surface.

It should be appreciated that the composition of oral care compositions (9) and (11) are duplicates of oral care compositions (8) and (10), respectively. Oral care compositions (9) and (11), however, were cast or aged under lower temperature conditions and lower casting times. As illustrated in Table 2, it was discovered that heating of oral care compositions at relatively higher temperatures during the casting and/or aging process resulted in reduced amounts of AO %. Without being bound by theory, it is believed that heating at least partially contributes to the evaporation of the peroxide source from the respective oral care compositions. In addition to the foregoing, it was discovered that reducing casting times resulted in increased amounts of AO. Without being bound by theory, it is believed that reducing the casting times also resulted in reduced evaporation of the peroxide source.

### Example 3

Exemplary oral care compositions (13)-(18) were prepared as solid films. To prepare the oral care compositions (13)-(18), respective slurries were prepared by combining the ingredients according to Table 3. Each of the slurries was then cast under varying casting conditions, as indicated in Table 3.

TABLE 3
Oral Care Composition (13)-(19)

| Component | (13) | (14) | (15) | (16) | (17) | (18) | (19) |
|---|---|---|---|---|---|---|---|
| Water (wt %) | 59.4 | 59.4 | 59.4 | 59.4 | 45 | 45 | 40.71 |
| Hydroxypropyl Cellulose (wt %) | — | — | — | — | — | — | — |
| High MW PVP (wt %) | 12 | 12 | 12 | 12 | 12 | 12 | — |
| Low MW PVP (wt %) | 12 | 12 | 12 | 12 | 12 | 12 | — |
| Cross-Linked PVP (wt %) | — | — | — | — | — | — | 21.27 |
| Propylene Glycol (wt %) | — | — | — | 12 | — | — | — |
| PEG 600 (wt %) | 12 | 12 | 12 | — | 12 | 12 | 8.68 |
| Sodium Lauryl Sulfate (wt %) | 1.2 | 1.2 | 1.2 | 1.2 | 1.2 | 1.2 | 2.04 |
| Betaine (wt %) | — | — | — | — | — | — | — |
| 50% HP Solution (wt %) | 3 | 3 | 3 | 3 | 3 | 3 | — |
| 18% PVP-H₂O₂ Solid | — | — | — | — | — | — | 23.52 |
| Sucralose (wt %) | 0.4 | 0.4 | 0.4 | 0.4 | 0.4 | 0.4 | 0.68 |
| Menthol (wt %) | — | — | — | — | — | — | — |
| Flavor (wt %) | | | | | | | 3.09 |
| Excipient (wt %) | Bal. | Bal. | Bal. | Bal. | Bal. | Bal. | Bal. |
| Casting Temperature (° C.) | 70 | 70 | 70 | — | 63 | 67 | 65 |
| Casting time (min) | 40 | 45 | 45 | — | 48 | 47 | 50 |
| Film Theoretical (H₂O₂%) | 4.06 | 4.31 | 4.26 | — | 3.443 | 3.366 | 5.0 |
| Film initial (H₂O₂%) | 3.07 | 2.58 | 2.89 | — | 3.17 | 2.98 | — |
| Film 1 wk at 60° C. 0% RH (H₂O₂%) | — | — | — | — | — | — | — |
| Film @ RT for 10 days | 2.38 | 2.54 | 1.99 | — | — | — | — |
| Film @ RT for 1 month | — | — | — | — | — | 2.834 | — |
| Film @ 40° C. for 1 month | — | — | — | — | — | 2.372 | — |
| Film @ 49° C. for 1 month | — | — | — | — | — | 2.518 | — |

### Example 4

Exemplary oral care compositions (20)-(23) including MPS as the peroxide source were prepared as solid films. To prepare the oral care compositions (20)-(23), respective slurries were prepared by combining the ingredients according to Table 4. Each of the slurries was then cast under varying casting conditions, as indicated in Table 4.

TABLE 4
Oral Care Composition (20)-(23)

| Component | (20) | (21) | (22) | (23) |
|---|---|---|---|---|
| Water | 59.35 | 59.35 | 59.35 | 59.35 |
| High MW PVP (wt %) | 12 | 12 | 12 | 12 |
| Low MW PVP (wt %) | 12 | 12 | 12 | 12 |
| Propylene Glycol (wt %) | 10 | 12.85 | 12.85 | 12.85 |
| Sodium Lauryl Sulfate | 1.2 | 1.2 | 1.2 | 1.2 |
| (wt %) | | | | |
| Sucralose (wt %) | 0.4 | 0.4 | 0.4 | 0.4 |
| Flavor (wt %) | Bal. | — | — | — |
| MPS (solid) (wt %) | 2.2 | 2.2 | 2.2 | 2.2 |
| Theoretical AO of Film (%) | 0.3035 | 0.296% | 0.296% | 0.296% |
| Film Initial AO (%) | 0.112 | 0.159% | 0.129% | 0.142% |
| Recovery of AO (%) | 36.86 | 53.7% | 43.5% | 48.2% |
| Casting Temperature (° C.) | 70 | 70 | 70-72 | 74 |
| Casting Time (min) | 60 | 30 | 40 | 54 |

As illustrated in Table 4, the oral care composition (20) included flavor while the oral care compositions (21)-(23) excluded flavor. As further indicated in Table 4, the amount of AO recovered in oral care composition (20) was relatively low at about 36.9%. Without being bound by theory, it is believed that the decreased amount of recovered AO was due, at least in part, to the presence of the flavorant in the oral care composition (20).

### Example 5

Exemplary oral care compositions (21)-(23) including MPS as the peroxide source were prepared as solid films. To prepare the oral care compositions (21)-(23), respective slurries were prepared by combining the ingredients according to Table 5. Each of the slurries was then cast under varying casting conditions and subsequently aged, as indicated in Table 5.

TABLE 5
Oral Care Composition (21)-(23)

| Component | (21) | (22) | (23) |
|---|---|---|---|
| Water (wt %) | 60.2 | 60.2 | 60.2 |
| High MW PVP (wt %) | 12 | 12 | 12 |
| Low MW PVP (wt %) | 12 | 12 | 12 |
| PEG 600 (wt %) | 12 | 12 | 12 |
| Sodium Lauryl Sulfate (wt %) | 1.2 | 1.2 | 1.2 |
| Sucralose (wt %) | 0.4 | 0.4 | 0.4 |
| MPS (solid) (wt %) | 2.2 | 2.2 | 2.2 |
| Theoretical AO of Film (%) | 0.277% | 0.277% | 0.277% |
| Film Initial AO (%) | 0.198% | 0.169% | n/a |
| Recovery of AO (%) | 71.5 | 61.1% | n/a |
| Casting Temperature (° C.) | 70 | 70 | 70 |
| Casting Time (min) | 45 | 45 | 45 |
| Film 1 wk @ RT (AO %) | 0.172% | n/a | 0.174% |
| Film 2 wk @ RT (AO %) | — | 0.142% | — |
| Film 1 mo. @ RT (AO %) | — | — | 0.128 |
| Film 2 mo. @ RT (AO %) | — | — | 0.105 |
| Film 3 mo. @ RT (AO %) | — | — | 0.107 |
| Film 1 mo. @ 40° C. (AO %) | — | — | 0.116 |
| Film 2 mo. @ 40° C. (AO %) | — | — | 0.069 |
| Film 3 mo. @ 40° C. (AO %) | — | — | 0.059 |
| Observations | Flexible | Flexible | Flexible |
| | Film | Film | Film |

As illustrated in Table 5, the oral care compositions (21)-(23) were compositionally the same and utilized PEG 600 as the plasticizer. It was observed that utilizing PEG 600 resulted in sufficient flexibility with the solid films. As illustrated in Table 5, the amount of AO recovered after 2 weeks of aging demonstrated the stability of the MPS in the prepared solid films.

### Example 6

Exemplary oral care compositions (24)-(26) including MPS as the peroxide source were prepared as solid films. To prepare the oral care compositions (24)-(26), respective slurries were prepared by combining the ingredients according to Table 6. Each of the slurries was then cast under varying casting conditions and subsequently aged, as indicated in Table 6.

TABLE 6
Oral Care Composition (24)-(26)

| Component | (24) | (25) | (26) |
|---|---|---|---|
| Water (wt %) | 57.55 | 58.4 | 60.2 |
| High MW PVP (wt %) | 12 | 12 | 0 |
| Low MW PVP (wt %) | 12 | 12 | 0 |
| Cross-Linked PVP (wt %) | 0 | 0 | 24 |
| Flavor (wt %) | 2.85 | 0 | 0 |
| PEG 600 (wt %) | 11 | 13 | 12 |
| Sodium Lauryl Sulfate (wt %) | 1.2 | 1.2 | 1.2 |
| Sucralose (wt %) | 0.4 | 0.4 | 0.4 |
| MPS (solid) (wt %) | 2.2 | 2.2 | 2.2 |
| Zinc Stearate (wt %) | 2 | 2 | 2 |
| Theoretical AO of Film (%) | 0.2325 | 0.2372 | 0.2372 |
| Film Initial AO (%) | 0.134 | 0.049 | 0.037 |
| Recovery of AO (%) | — | — | — |
| Casting Temperature (° C.) | 70 | 78 | 70 |
| Casting Time (min) | 45 | 31 | 38 |

As illustrated in Table 6, the oral care composition (26) included cross-linked PVP as the thickening agent in lieu of the high and low molecular weight PVP. Cross-linked PVP was utilized to improve the handling of the solid films. It was observed, however, that cross-linked PVP resulted in a relatively “sticky” or tacky solid film.

As illustrated in Table 6, zinc stearate was included in the oral care compositions (24)-(26) as a releasing agent to facilitate the preparation of the solid films. As further illustrated, oral care composition (24) included flavor and oral care composition (25) did not include flavor.

### Example 7

Several releasing agents and substrates were utilize for varying oral care compositions including either MPS or a hydrogen peroxide solution. Observations are summarized in Table 7.

TABLE 7
Observations For Combining Releasing Agents/Substrates
and MPS or H₂O₂ Solution

| Releasing Agent | H₂O₂ Solution | MPS |
|---|---|---|
| 30% Betaine water | H₂O₂ unstable | MPS unstable; cannot |
| solution | | peel sufficiently |
| Polysorbate 80/20 | Cannot sufficiently peel | Cannot sufficiently peel |
| 29% SLS | Cannot sufficiently peel | Cannot sufficiently peel |
| Pure silicon oil | Too hydrophobic, films | Too hydrophobic, films |
| | are not even | are not even |
| Zinc Stearate | — | MPS unstable |
| Non-stick Silicon | Good film; Peels well | Good film; Peels well |
| Film/Sheet | | |
| Mylar Film | — | Cannot sufficiently peel |

### Example 8

Exemplary oral care compositions (27) and (28) including MPS as the peroxide source were prepared as solid films. To prepare the oral care compositions (27) and (28), respective slurries were prepared by combining the ingredients according to Table 8. Each of the slurries was then cast under varying casting conditions and subsequently aged, as indicated in Table 8.

TABLE 8
Oral Care Composition (27) and(28)

| Component | (27) | (28) |
|---|---|---|
| Water (wt %) | 45.2 | 45.2 |
| High MW PVP (wt %) | 12 | 12 |
| Low MW PVP (wt %) | 12 | 12 |
| PEG 600 (wt %) | 12 | 12 |
| Sodium Lauryl Sulfate (wt %) | 1.2 | 1.2 |
| Sucralose (wt %) | 0.4 | 0.4 |
| MPS (solid) (wt %) | 2.2 | 2.2 |
| Casting Temperature (° C.) | 60 | 60 |
| Casting Time (min) | 60 | 60 |
| Theoretical AO of Film (%) | 0.228 | 0.201 |
| Film Initial AO (%) | 0.192 | 0.176 |
| Recovery of AO (%) | 83.8 | 87.8 |
| Film @ RT for 1 month (AO %) | 0.122 | 0.122 |
| Film @ RT for 2 month (AO %) | | 0.111 |
| Film @ RT for 3 month (AO %) | | 0.084 |
| Film @ 40° C. for 1 month (AO %) | 0.053 | 0.053 |
| Film @ 40° C. for 2 month (AO %) | | 0.039 |
| Film @ 40° C. for 3 month (AO %) | | 0.045 |

It should be appreciated that oral care compositions (27) and (28) utilized about 15% less water than previous oral care compositions prepared. As compared to oral care compositions (21) and (22) of Table 5, the oral care compositions (27) and (28) were cast at lower temperatures for a longer period of time. Further, oral care compositions (21) and (22) included less water in the films in order to alter the thickness during casting. It was discovered that the casting conditions and/or the composition having lower water content resulted in relatively more stable MPS.

As further illustrated in Table 8, oral care composition (33) utilized only high molecular weight PVP. It was surprisingly and unexpectedly discovered that utilizing only the high molecular weight PVP resulted in solid films that were no sufficiently flexible and brittle. Without being bound by theory, it was believed that a combination of the high and low molecular weight PVP in the proper ratios were needed to adjust the glass transition temperature below room temperature.

### Example 9

An exemplary oral care compositions (29)-(30) including a plurality of layers were prepared and evaluated for their respective stability. To prepare the oral care compositions (29)-(30), a base layer was prepared by combining the ingredients/components according to Table 9.

TABLE 9
Base Layer Composition

| Component | (wt %) |
|---|---|
| Water (wt %) | 35.2 |
| High MW PVP (wt %) | 12 |
| Low MW PVP (wt %) | 12 |
| Flavor | 2.8 |
| PEG 600 (wt %) | 12 |
| Sodium Lauryl Sulfate (wt %) | 1.2 |
| Sucralose (wt %) | 0.4 |
| Casting Temperature (° C.) | 60 |
| Casting Time (min) | 30 |
| Casting Substrate | Non-stick Silicone Surface |

The oral care composition (29) was prepared as a two-layered film. The base layer was prepared according to Table 9. After the base layer was dried, a layer of a 20% MPS aqueous solution (6 g total) was applied or contacted with the base layer and dried at about 60° C. for about 10 min.

The oral care composition (30) was prepared as a three-layered film. The base layer was prepared according to Table 9. A second layer was prepared by applying or contacting a block co-polymer of ethylene oxide and propylene oxide with the base layer using a roller. Specifically, PLURACARE® L1220, a block co-polymer of ethylene oxide and propylene oxide, was applied to the base layer using a roller. The block co-polymer was used as received. The block co-polymer was utilized, in part, to provide a tacky surface. A third layer adjacent the second layer was prepared by dusting, applying, or contacting MPS powder on the second layer using a 100 screen mesh. Each of the oral care compositions (29) and (30) were then evaluated for stability under accelerated aging conditions. The results are summarized in Table 10.

TABLE 10
Oral Care Composition (29)-(30)

| | Two-Layer | Three-Layer |
|---|---|---|
| Conditions | (29) | (30) |
| Theoretical AO of Film (%) | 0.414% | 2.295% |
| Film Initial AO (%) | 0.428% | 2.463% |
| Film 1 wk @ RT (AO %) | 0.262% | 1.983% |
| Film 3 wk @ RT (AO %) | 0.282% | 1.689% |
| Film 1 wk. @ 40° C. (AO %) | 0.163% | 2.014% |
| Film 3 wk. @ 40° C. (AO %) | 0.291% | 1.227% |

It is noted that the variability in the oral care compositions (29) and (30) may be due in part to the non-uniformity of applying the active layer including the MPS. It should be appreciated that while the application of the block co-polymer in the oral care composition (30) provided a tacky surface, the application of the third layer of the MPS powder eliminated the tackiness of the film, and the tackiness was not present after aging, thereby demonstrating the stability of the multilayered films. It was further surprisingly and unexpectedly discovered that utilizing the multi-layered films improved the stability of the oral care compositions. Particularly, the single layer solid films prepared herein exhibited degradation of about 50% or more, and utilizing the multilayer films reduced the degradation to about 40%.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

What is claimed is:

1. An oral care composition, comprising an orally acceptable vehicle and one or more whitening agents, wherein the oral care composition is a toothpaste in the form of a solid film, and wherein the one or more whitening agents comprise at least one of a source of hydrogen peroxide, a non-peroxide bleaching agent, or a combination thereof, and wherein the solid film is dissolvable and/or disintegrable.

2. The oral care composition of claim 1, wherein the whitening agent comprises the source of hydrogen peroxide, and wherein the source of hydrogen peroxide comprises a hydrogen peroxide solution.

3. The oral care composition of claim 1, wherein the whitening agent comprises the source of hydrogen peroxide, and wherein the source of hydrogen peroxide is a solid, wherein the source of hydrogen peroxide comprises one or more of a cross-linked polyvinylpyrrolidone (PVP) hydrogen peroxide complex, a polyvinylpyrrolidone (PVP) hydrogen peroxide complex, carbamide peroxide, a percarbonate, a hydrogen peroxide—silica complex or a combination thereof.

4. The oral care composition of claim 1, wherein the source of hydrogen peroxide is present in an amount sufficient to provide free hydrogen peroxide in an amount of from about 0.01 weight % to less than or equal to about 10 weight %, based on the total weight of the oral care composition.

5. The oral care composition of claim 1, wherein the whitening agent comprises the non-peroxide bleaching agent.

6. The oral care composition of claim 5, wherein the non-peroxide bleaching agent comprises a salt of monopersulfate (MPS).

7. The oral care composition of claim 6, wherein the salt of MPS comprises one or more of potassium MPS, sodium MPS, ammonium MPS, or a combination thereof.

8. The oral care composition of claim 6, wherein the salt of MPS comprises 2KHSO₅·KHSO₄·K₂SO₄.

9. The oral care composition of claim 1, wherein the orally acceptable vehicle comprises a polymer base comprising one or more water soluble polymers.

10. The oral care composition of claim 9, wherein the one or more water soluble polymers comprise one or more of carboxymethyl cellulose (CMC), hydroxyethyl cellulose (HEC), cetyl hydroxyethylcellulose, hydroxypropyl methylcellulose (HPMC), hydroxypropyl cellulose (HPC), polyvinylpyrrolidone (PVP), polyvinylpyrrolidone/vinyl acetate (PVP/VA), cellulose ethers, or a combination thereof.

11. The oral care composition of claim 10, wherein the one or more water soluble polymers comprise polyvinylpyrrolidone (PVP, wherein the PVP comprises a combination of high molecular weight PVP and low molecular weight PVP.

12. The oral care composition of claim 11, wherein the high molecular weight PVP comprises a molecular weight of from about 200,000 Da to about 1,500,000 Da, or about 300,000 Da to about 1,300,000 Da, or from 500,000 Da to about 1,000,000 Da, and wherein the low molecular weight PVP comprises a molecular weight of from about 10,000 Da to about 100,000 Da, about 30,000 Da to about 70,000 Da, or about 40,000 Da to about 60,000, Da.

13. The oral care composition of claim 11, wherein a weight ratio of the high molecular weight PVP to the low molecular weight PVP is from about 0.5:1 to about 2:1.

14. The oral care composition of claim 1, further comprising one or more plasticizers, wherein the plasticizer comprises one or more of glycerin, sorbitol, an alkylene glycol, or a combination thereof.

15. The oral care composition of claim 14, wherein the plasticizer comprises the alkylene glycol, wherein the alkylene glycol comprises one or more of polyethylene glycol, polypropylene glycol, or a combination thereof.

16. The oral care composition of claim 1, wherein the oral care composition is free or substantially free of menthol, betaine, fluoride containing components, or a combination thereof.

17. The oral care composition of claim 1, further comprising one or more anticalculus agents, optionally, the anticalculus agents comprises one or more phosphates, polyphosphates, or a combination thereof, further optionally, the anticalculus agents comprises one or more of tetrasodium pyrophosphate (TSPP), sodium tripolyphosphate (STPP), tetrapotassium pyrophosphate (TKPP), or a combination thereof.

18. The oral care composition of claim 1, wherein the solid film comprises a plurality of layers, optionally, a first layer of the plurality of layers comprises the orally acceptable vehicle, further optionally, a second layer of the plurality of layers comprises the one or more whitening agents.

19. The oral care composition of claim 18, further comprising a third layer interposed between the first layer and the second layer, wherein the third layer is an adhesive layer.

20. The oral care composition of claim 18, wherein the second layer is disposed adjacent the first layer.

21. The oral care composition of claim 1, further comprising an abrasive, optionally, the abrasive comprises an insoluble phosphate salt, further optionally, the abrasive is present in an amount of from about 0.1 wt % to about 20 wt %, based on the total weight of the oral care composition.

22. A method for preparing the oral care composition of claim 1, the method comprising:
contacting the orally acceptable vehicle, the one or more whitening agents, and water with one another to prepare a slurry; and
preparing the solid film with the slurry.

23. The method of claim 22, wherein preparing the solid film with the slurry comprises casting at a temperature of from about 25° C. to about 80° C. or less, 75° C. or less, 70° C. or less, 65° C. or less, 60° C. or less, 50° C. or less, or 40° C. or less.
